(19) United States
(12) Patent Application Publication
(10) Pub. No.: US 2025/0312266 A1
(43) Pub. Date: Oct. 9, 2025

(54) COSMETIC USE OF PARTIALLY OR TOTALLY HYDROGENATED POLYFARNESENES, AND METHOD FOR THE COSMETIC TREATMENT OF HAIR

(71) Applicants: SOCIETE D'EXPLOITATION DE PRODUITS POUR LES INDUSTRIES CHIMIQUES SEPPIC, PARIS CEDEX 07 (FR); TOTALENERGIES ONETECH, COURBEVOIE (FR)

(72) Inventors: Elodie CUQ ARNAUD, CASTRES (FR); Jennifer FRYSCHER, LA GARENNE COLOMBES (FR)

(21) Appl. No.: 18/849,792

(22) PCT Filed: Mar. 23, 2023
(86) PCT No.: PCT/EP2023/057436

§ 371 Date: Sep. 23, 2024

(30) Foreign Application Priority Data
Mar. 24, 2022 (FR) FR2202634

(51) Int. Cl. A61K 8/81; A45D 2/00; A61Q 5/06

(52) U.S. Cl. CPC A61K 8/8194; A45D 2/00; A61Q 5/06

(57) ABSTRACT

The invention relates to the use of a composition (C) comprising, for 100% of the weight thereof,—from 10% by weight to 90% by weight of at least one poly(farnesene) polymer having a number-average molecular weight of greater than or equal to 10,000 g/mol and less than or equal to 120,000 g/mol, said poly(farnesene) polymer being partially or totally hydrogenated and/or functionalized with hydroxyl radicals, and—from 10% by weight to 90% by weight of a mixture (M) of hydrocarbons in which at last 94% by weight of said hydrocarbons comprise from 15 to 19 carbon atoms, for preventing and/or slowing the appearance of unattractive effects on human hair following exposure to intense heat. The invention also relates to a method for drying and shaping the hair, using said composition.

### TECHNICAL FIELD OF THE INVENTION

The present invention relates to the field of cosmetics. More specifically, it relates to the cosmetic treatment of hair.

### TECHNOLOGICAL BACKGROUND

When drying or styling, hair can be exposed to the heat of hot air from hair dryers or heat from curling tongs or straightening irons, or else heat from heated combs or brushes. This exposure causes heat stress that can cause modifications to the polypeptide chains of the hair's keratin, more particularly affecting their stereochemical conformations and certain intermolecular bonds, and consequently adversely affecting the internal structure of the hair. Such adverse effects lead to both a deterioration in the appearance of the hair, for example a reduction in glossiness, and a deterioration in the sensory properties of the hair, for example reduced softness. In addition, external hair damage is sometimes observed, such as cuticle lifting and/or blistering of individual hair fibers, which weakens the hair by damaging its surface, or even causing it to break, due to the resulting increase in friction between hair fibers.

Numerous compositions have been developed and sold to protect hair from the negative effects associated with exposing the hair to high temperatures. They form a coating on the hair fibers, thereby reducing the effect of heat within said fibers. These compositions comprise film-forming polymers, such as those resulting from the polymerization of N-vinylpyrrolidones, vinyl alcohols or vinyl acetate.

The international application published under number WO 2014/95210A1 discloses a composition comprising at least one non-ionic polymer, such as a mixture of copolymers derived from N-vinylpyrrolidones and/or vinyl acetate, with at least one cationic polymer, for example Polyquaternium-46. The presence of the cationic polymer reduces or eliminates the sticky sensation caused by the film-forming polymer and limits the presence of unsightly white residues on the hair.

Silicone derivatives are also known as multifunctional ingredients in hair care compositions to prevent heat-generated damage, among other properties. For example, there are “aminoglycol” type copolymers such as bis(C13-C15 alkoxy) PG-amodimethicone, sold under the name: “Dow Corning 8500 conditioning agent”. Hair care compositions containing them are stable, spread easily over the hair and form a protective film that prevents water loss from the hair shaft due to exposure to heat.

Polymers of the siloxane family are also known to protect hair exposed to heat. These include, for example, poly(dimethylsiloxane) of formula: —[O—Si(CH₃)₂]ₙ—, commonly known as PDMS or dimethicone, or dimethiconols, which are characterized by a structure of formula: HO— Si(CH₃)₂—[O—Si(CH₃)₂]ₙ—Si(CH₃)₂—OH. Mixtures of dimethicone and dimethiconol are widely used in hygiene formulas (especially shampoos) and hair care formulas.

Silicone compounds that protect hair exposed to heat also include those of the aminosilane family, such as those described in the French patent application published under number FR 3,038,513 A1, and the US patent application published under number US 2018/161266 A2. The international application published under number WO 2021/260486 A1, for example, discloses compositions comprising a film-forming polymer selected from Polysilicone-35, Polysilicone-33, Polysilicone-29, Polysilicone-28, Polysilicone-25, Polysilicone-24, Polysilicone-22, Polysilicone-19, Polysilicone-18, Polysilicone-14 and Polysilicone-3, alone or as mixtures, and a silane derivative of a hydrolyzed protein or of a peptide or of an amino acid.

However, the above-mentioned polymers have little or no biodegradability and are derived from raw materials of fossil origin.

The inventors therefore set out to develop a novel alternative solution to the above-mentioned copolymers which, while providing sufficient protection for hair when exposed to heat, has environmental characteristics in line with both current and future regulations and consumer requirements.

### DISCLOSURE OF THE INVENTION

According to a first aspect, the subject of the invention is the use of a composition (C) comprising, for 100% of the weight thereof,
from 10% by weight to 90% by weight of at least one poly(farnesene) polymer having a number-average molecular weight of greater than or equal to 10,000 g/mol and less than or equal to 120,000 g/mol, said poly(farnesene) polymer being partially or totally hydrogenated and/or functionalized with hydroxyl radicals, and
from 10% by weight to 90% by weight of a mixture (M) of hydrocarbons in which at least 94% by weight of said hydrocarbons comprise from fifteen to nineteen carbon atoms, for preventing and/or slowing the appearance of unattractive effects on human hair following exposure to intense heat.

For the purposes of the present invention, “unattractive effects on human hair” means any change in external appearance due to the consequences of exposure to intense heat, such as cuticle lifting, blistering of individual hair fibers, dulling, weakening of hair with respect to mechanical stresses, the appearance of split ends, cracks and breaks in the hair, loss of softness, increased porosity of the hair fiber and/or reduced hydrophobicity of the hair.

For the purposes of the present invention, “intense heat” refers to the induced heat emitted by appliances used to dry, straighten and/or curl wet hair, such as hair dryers, drying hoods, heated combs, heated hairbrushes, straightening irons and curling tongs. This “intense” heat is emitted when these appliances are operated at a temperature generally of between 50° C. and 250° C.

In the context of the present invention, the poly(farnesene) polymer(s) contained in composition (C) as defined above are known and their chemical structure is detailed in the international application published under number WO 2020/144440 A1. They can be obtained by the method described in the international application published under number WO 2018/052709 A1.

In the context of the present invention, “mixture (M) of hydrocarbons” means a mixture of branched alkanes and/or linear alkanes and/or cycloalkanes. “Linear alkanes” comprising from fifteen to nineteen carbon atoms denotes n-pentadecane, n-hexadecane, n-heptadecane, n-octadecane and n-nonadecane; “branched alkanes” (or isoalkanes) comprising from fifteen to nineteen carbon atoms denotes in particular 2-methyltetradecane (or isopentadecane), 2-methylpentadecane (or isohexadecane), 2-methylhexadecane (or isoheptadecane), 2-methylheptadecane (or iso-octadecane) and 2-methyloctadecane (or isononadecane); “cycloalkanes comprising from fifteen to nineteen carbon atoms” denotes more particularly saturated hydrocarbons comprising at least one saturated cyclic hydrocarbon group optionally substituted with one or more linear or branched alkyl radicals. These mixtures can be obtained by conversion of a biomass, as disclosed in WO 2020/144440 A1.

According to a particular embodiment of the present invention, the mixture (M) contained in the composition (C) as defined above comprises, for 100% of the weight thereof:
from 15% to 20% by weight of alkanes (linear, isoalkanes and cycloalkanes) comprising 17 carbon atoms,
from 70% to 75% by weight of alkanes (linear, isoalkanes and cycloalkanes) comprising 18 carbon atoms, and
from 4% to 6% by weight of alkanes (linear, isoalkanes and cycloalkanes) comprising 19 carbon atoms.

According to a more particular embodiment of the present invention, the mixture (M) contained in the composition (C) as defined above is a mixture of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
15% to 20% by weight of alkanes (linear, isoalkanes and cycloalkanes) comprising 17 carbon atoms,
70% to 75% by weight of alkanes (linear, isoalkanes and cycloalkanes) comprising 18 carbon atoms, and
4% to 6% by weight of alkanes (linear, isoalkanes and cycloalkanes) comprising 19 carbon atoms.

In the context of the present invention, the poly(farnesene) polymer(s) contained in the composition (C) as defined above more particularly have a number-average molecular weight of greater than or equal to 20,000 g/mol and less than or equal to 85,000 g/mol, even more particularly of greater than or equal to 30,000 g/mol and less than or equal to 80,000 g/mol.

According to a very particular embodiment of the present invention, the poly(farnesene) polymer contained in the composition (C) as defined above has a number-average molecular weight of 71,000 g/mol and is totally hydrogenated and functionalized with hydroxyl radicals.

The subject of the invention is more particularly the use as defined above, characterized in that said composition (C) comprises, for 100% of the weight thereof,
40% by weight of a totally hydrogenated poly(farnesene) polymer functionalized with hydroxyl groups and with hydroxyl groups and having a number-average molecular weight equal to 71,000, and
60% by weight of a mixture (M) of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms.

Composition (C) as defined above can be prepared by simply mixing said poly(farnesene) polymer and the mixture (M) at a temperature of between 20° C. and 60° C. with mechanical stirring provided by an anchor or turbine-type spindle at a speed of between 20 rpm and 500 rpm.

According to another particular aspect, a subject of the invention is the use as defined previously, characterized in that said composition (C) is implemented in a cosmetic formulation (F) comprising from 0.2% to 40% by weight to 40% by weight thereof for 100% of the weight thereof; and more particularly the use as defined above, characterized in that said formulation (F) is an emulsion (E) of an aqueous phase (A) and a fatty phase (G) comprising, for 100% of the weight thereof:
from 50% to 98% by weight of said aqueous phase (A), and
from 2% to 50% by weight of said fatty phase (G), said fatty phase (G) comprising, for 100% of the weight thereof:
from 10% to 80% by weight of said fatty composition (C),
from 0.5% to 20% by weight of an emulsifying surfactant (S) or a mixture of emulsifying surfactants (Ms) of the oil-in-water or water-in-oil type, and
from 0% to 89.5% by weight of an oil or a plurality of oils and/or a wax, said wax being different from said composition (C).

According to this particular aspect, the oil(s) optionally contained in the fatty phase (G) as defined above are chemical substances or mixtures of chemical substances which are insoluble in water and which have a liquid appearance at a temperature of 25° C. These oils can be of animal or plant origin, or be synthetic oils.

An example of an oil of animal origin implemented in the context of the present invention is squalene or squalane.

Examples of oils of plant origin implemented in the context of the present invention include phytosqualane, sweet almond oil, coconut oil, castor oil, jojoba oil, olive oil, rapeseed oil, peanut oil, sunflower oil, wheat germ oil, corn germ oil, soybean oil, cottonseed oil, alfalfa oil, poppy seed oil, pumpkin oil, evening primrose oil, millet oil, barley oil, rye oil, safflower oil, candlenut oil, passion flower oil, hazelnut oil, palm oil, shea butter, apricot kernel oil, calophyllum oil, sisymbrium oil, avocado oil, calendula oil, sesame oil, meadowsweet oil, macadamia kiwi oil, borage oil, blackcurrant seed oil, coffee oil, pistachio oil, peach kernel oil, raspberry seed oil, strawberry seed oil, melon oil, blueberry seed oil, argan oil, plum oil extract, pomegranate oil, papaya oil, coconut milk oil, flower or vegetable oils and ethoxylated vegetable oils. Examples of synthetic oils implemented in the context of the present invention include fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate; esters derived from lanolic acid, such as isopropyl lanolate, isocetyl lanolate, glycerol triheptanoate, alkylbenzoates, hydrogenated oils, polyalphaolefins, polyolefins such as polyisobutene, hydrogenated polydecene or hydrogenated polyisobutene, sold in France by Ets B. Rossow et Cie under the name PARLEAM—POLYSYNLANE™, cited in: Michel and Irene Ash; Thesaurus of Chemical Products, Chemical Publishing Co, Inc. 1986 Volume I, page 211 (ISBN 0 7131 3603 0); silicone oils such as dimethylpolysiloxanes, methylphenylpolysiloxanes, amine-modified silicones, fatty acid-modified silicones, alcohol-modified silicones, silicones modified by alcohols and fatty acids, silicones modified by polyether groups, modified epoxy silicones, silicones modified by fluoro groups, cyclic silicones and silicones modified by alkyl groups.

According to this particular aspect, the wax optionally included in the fatty phase (G) as defined above is a chemical substance or a mixture of chemical substances which are insoluble in water and which have a solid appearance at a temperature of 45° C. Examples include beeswax, camauba wax, candelilla wax, ouricury wax, Japan wax, cork fiber wax, sugar cane wax, jojoba wax, blackcurrant blossom wax, narcissus blossom wax, orange blossom wax, orange wax, rice wax, lignite waxes, microcrystalline waxes, lanolin wax; ozokerite; polyethylene wax; silicone waxes; vegetable waxes; fatty alcohols and fatty acids which are solid at room temperature and glycerides which are solid at room temperature.

According to this particular aspect, and depending on the nature of the emulsifying surfactant (S) or of the mixture of emulsifying surfactants (Ms), the emulsion (E) as defined above is either an oil-in-water (O/W) type emulsion or a water-in-oil (W/O) type emulsion.

Emulsifying surfactant (S₁) of the oil-in-water type, or mixture of emulsifying surfactants (Ms₁) of the oil-in-water type, present in the fatty phase (G₁) of the emulsion (E₁) of (O/W) type, denotes a chemical substance or a mixture of chemical substances which makes it possible to stabilize the droplets of said fatty phase (G₁) dispersed in the continuous aqueous phase (A). Examples of said emulsifying surfactant (S₁) or of said mixture of emulsifying surfactants (Ms₁) include in particular:
Polysorbates resulting from the ethoxylation reaction between one molar equivalent of sorbitan esters, such as sorbitan laurate, sorbitan palmitate, sorbitan stearate, sorbitan isostearate or sorbitan oleate with 5 to 20 molar equivalents of ethylene oxide;
Products resulting from the ethoxylation reaction between one molar equivalent of a fatty acid, such as palmitic acid, myristic acid, lauric acid, stearic acid, isostearic acid or oleic acid, with 5 to 40 molar equivalents of ethylene oxide;
Products resulting from the esterification reaction between a fatty acid, such as palmitic acid, myristic acid, lauric acid, stearic acid, isostearic acid, oleic acid, arachidic acid or behenic acid with 3 to 20 molar equivalents of glycerol;

Emulsifying surfactants of water-in-oil type (S₂) which can be used in the context of the present invention include in particular:
lipoamino acids and salts thereof and lipopeptides and salts thereof; sorbitan esters such as sorbitan laurate, palmitate, stearate, oleate, sesquioleate, trioleate and isostearate; glycerol esters such as glycerol laurate, palmitate, stearate, oleate, sesquioleate, trioleate and isostearate;
esters of polyglycol, polyglycerol or polyols such as polyglycol or polyglycerol laurate, palmitate, stearate, oleate, sesquioleate, trioleate, isostearate, polyhydroxystearates, such as HYPERMER™ B246, ARLACEL™ P135, DEHYMULS™ PGPH and DECAGLYN™ 5HS,
polyethylene glycol-alkyl glycol copolymers such as PEG-45 dodecyl glycol copolymer such as ELFACOS ST 9™;
sucrose esters, ethoxylated or non-ethoxylated methylglucoside esters,
fatty acids and ethoxylated fatty alcohols;
anionic emulsifiers such as decyl phosphate or cetearyl sulfate; aluminum polyoxystearate, such as MANALOX®, magnesium stearate and aluminum stearate; and
alkylpolyglycosides, compositions of alkylpolyglycosides and of fatty alcohols.

An example of alkylpolyglycoside compositions is the composition (CA₁) represented by the formula: R₁—O-(G)ₓ-H in which G represents the residue of a reducing sugar chosen from glucose or xylose, R₁—O— is bonded to G by the anomeric carbon of the saccharide residue so as to form an acetal function, x represents a decimal number of greater than or equal to 1.05 and less than or equal to 2.5, and R₁ represents an aliphatic radical chosen from n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl and 2-decyltetradecyl, said composition (CA₁) consisting of a mixture of compounds represented by the formulas R₁—O-(G)₁-H, R₁—O-(G)₂-H, R₁—O-(G)₃-H and R₁—O-(G)₄-H and R₁—O-(G)₅-H, in the respective molar proportions a₁, a₂, a₃, a₄ and a₅, such that the sum a₁+a₂+a₃+a₄+a₅ is equal to 1 and the sum a₁+2a₂+3a₃+4a₄+5a₅ is equal to x.

An example of compositions of alkylpolyglycosides and fatty alcohols is the composition (CA₂) comprising, for 100% of the weight thereof:
From 10% to 50% by weight and more particularly from 20% to 30% by weight of said composition (CA₁) as defined above, and
From 90% to 50% by weight and more particularly from 80% to 70% by weight of at least one fatty alcohol of formula: R′₁—OH, in which R′₁, which is identical to or different from R₁, represents a radical chosen from n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl or 2-decyltetradecyl radicals; R′₁ more particularly represents the 2-octyldodecyl radical.

Another example of compositions of alkylpolyglycosides and fatty alcohols is the composition (CA₃) comprising, for 100% of the weight thereof:
from 15% to 25% by weight of the composition (CA₁),
from 55% to 65% by weight of at least one fatty alcohol of formula R′₁—OH in which R′₁ represents the 2-octyldodecyl radical; and
from 10% to 30% by weight of at least one polyglycol poly(hydroxystearate) represented by the formula Z₂—O—[CH₂—CH(R₄)—O]_{y2}—Z′₂, in which y2 represents an integer greater than or equal to 2 and less than or equal to 50, R₄ represents the hydrogen atom, the methyl radical or the ethyl radical, Z₂ represents a radical of formula:

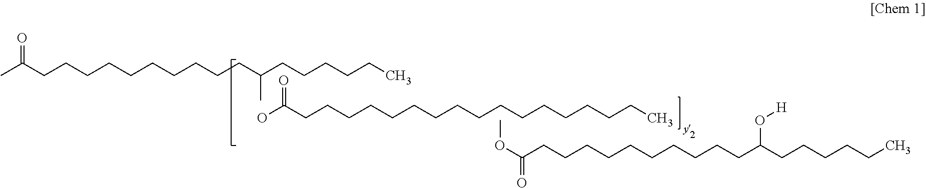

in which y′₂ represents an integer greater than or equal to 0 and less than or equal to 10, more particularly greater than or equal to 1 and less than or equal to 10, and Z′₂, which is identical to or different from Z₂, represents the hydrogen atom or the radical having the formula as defined above.

As the emulsifying surfactant (S₂) of (W/O) type or mixture of emulsifying surfactants (Mₛ₂) of (W/O) type present in the fatty phase (G₂) of an emulsion (E₂) of (W/O) type, there are more particularly alkylpolyglycoside compositions, compositions of alkylpolyglycosides and fatty alcohols, polyglycerol esters, alkoxylated polyglycerol esters, polyglycol polyhydroxystearates, polyglycerol polyhydroxystearates, alkoxylated polyglycerol polyhydroxystearates, polyethylene glycol-alkylglycol copolymers.

Said emulsion (E₂) of (W/O) type as defined above may also comprise, within said aqueous phase (A), for 100% of the weight thereof, from 0.5% by weight to 10% by weight and preferably from 0.5% by weight to 5% by weight of a crosslinked anionic polyelectrolyte (P) or a mixture of crosslinked anionic polyelectrolytes (Mₚ).

In the previous definition, crosslinked anionic polyelectrolyte (P) denotes a non-linear, crosslinked anionic polyelectrolyte in the form of a three-dimensional network which is insoluble in water, but is swellable with water and leads to the formation of a chemical gel.

Examples of said crosslinked anionic polyelectrolyte (P) are:
a homopolymer of 2-methyl-2-[(1-oxo-2-propenyl)amino]-1-propanesulfonic acid, partially or totally salified in sodium salt or ammonium salt form, crosslinked with triallylamine and/or methylenebis(acrylamide);
a copolymer of 2-methyl-2-[(1-oxo-2-propenyl)amino]-1-propanesulfonic acid (γ), partially or totally salified in sodium salt or ammonium salt form, and of acrylic acid (δ), partially or totally salified in sodium salt or ammonium salt form, crosslinked with triallylamine and/or methylenebis(acrylamide); in particular said copolymer in which the molar proportions of monomers (γ) and (δ) are as follows:

$$30 \% \leq ( γ ) \leq 90 \% or more particularly 40 \% \leq ( γ ) \leq 90 \% , and$$

$$10 \% \leq ( δ ) \leq 70 \% or more particularly 10 \% \leq ( δ ) \leq 60 \% ;$$

a copolymer of 2-methyl-2-[(1-oxo-2-propenyl)amino]-1-propanesulfonic acid (γ), partially or totally salified in sodium salt or ammonium salt form, and of acrylamide (ε), crosslinked with triallylamine and/or methylenebis(acrylamide); in particular said copolymer in which the molar proportions of monomers (γ) and (ξ) are as follows:

$$30 \% \leq ( γ ) \leq 90 \% , and$$

$$10 \% \leq ( ɛ ) \leq 70 \% ;$$

a copolymer of 2-methyl-2-[(1-oxo-2-propenyl)amino]-1-propanesulfonic acid (γ), partially or totally salified in sodium salt form, and of hydroxyethyl acrylate (ξ), crosslinked with triallylamine and/or methylenebis(acrylamide); in particular said copolymer in which the molar proportions of monomers (γ) and (ξ) are as follows:

$$30 \% \leq ( γ ) \leq 90 \% , and$$

$$10 \% \leq ( ξ ) \leq 70 \% ;$$

a terpolymer of 2-methyl-2-[(1-oxo-2-propenyl)amino]-1-propanesulfonic acid (γ), partially or totally salified in sodium salt or ammonium salt form, of acrylic acid (δ), partially or totally salified in sodium salt or ammonium salt form, and of acrylamide (ε), crosslinked with triallylamine and/or methylenebis(acrylamide); in particular said terpolymer in which the molar proportions of monomers (γ), (δ) and (ε) are as follows:

$$30 \% \leq ( γ ) \leq 45 \% , \\ 2 \% \leq ( δ ) \leq 10 \% ; and more particularly 2 \% \leq ( δ ) \leq 8 \% , and$$

$$45 \% \leq ( ɛ ) \leq 68 \% , and more particularly 47 \% \leq ( δ ) \leq 68 \% ;$$

a terpolymer of 2-methyl-2-[(1-oxo-2-propenyl)amino]-1-propanesulfonic acid (γ), partially or totally salified in sodium salt or ammonium salt form, of N,N dimethylacrylamide (β) and of tetraethoxylated lauryl methacrylate (α), crosslinked with trimethylolpropane triacrylate and/or triallylamine and/or methylenebis(acrylamide); in particular said copolymer in which the molar proportions of monomers (γ), (β) and (α) are as follows:

$$60 \% \leq ( γ ) \leq 80 \% , \\ 15 \% \leq ( β ) \leq 39.5 \% , and$$

$$0.5 \% \leq ( α ) \leq 5 \% ;$$

a tetrapolymer of 2-methyl-2-[(1-oxo-2-propenyl)amino]-1-propanesulfonic acid (γ), partially or totally salified in sodium salt or ammonium salt form, of N,N dimethylacrylamide (β), of lauroyl methacrylate (η) and of stearoyl methacrylate (μ), crosslinked with trimethylolpropane triacrylate and/or triallylamine and/or methylenebis(acrylamide); in particular said copolymer in which the molar proportions of monomers (γ), (β), (η) and (μ) are as follows:

$$60 \% \leq ( γ ) \leq 80 \% , \\ 15 \% \leq ( β ) \leq 39 \% , \\ 0.5 \% \leq ( η ) \leq 2.5 \% , and$$

$$0.5 \% \leq ( μ ) \leq 2.5 \% .$$

The emulsions (E), (E1) and (E₂) can also comprise excipients and/or active ingredients which are customarily used in the field of formulations intended to be applied to the hair or scalp. Therefore, according to another particular embodiment of the present invention, the emulsions (E), (E₁) and (E₂) as defined above additionally comprise one or more auxiliary compounds chosen from foaming and/or detergent surfactants, thickening and/or gelling surfactants, thickening and/or gelling agents, stabilizers, film-forming compounds, solvents and cosolvents, hydrotropic agents, plasticizing agents, emulsifying and co-emulsifying agents, opacifying agents, pearlizing agents, superfatting agents, sequestrants, chelating agents, antioxidants, fragrances, essential oils, preservatives, conditioning agents, deodorants, bleaching agents intended for bleaching body hair and skin, active ingredients for treating and/or protecting the skin or hair, sunscreens, mineral fillers or pigments, particles providing a visual effect or intended to encapsulate active ingredients, exfoliating particles, texturizing agents, optical brighteners or insect repellents.

Examples of foaming and/or detergent surfactants which are optionally present in the emulsions (E), (E₁) and (E₂) as defined above are topically acceptable anionic, cationic, amphoteric or non-ionic foaming and/or detergent surfactants which are customarily used in this field of cosmetics.

“Cosmetically acceptable” anionic foaming and/or detergent surfactants include salts of alkali metals, alkaline-earth metals or ammonium, amines, amino alcohols, alkyl ether sulfates, alkyl sulfates, alkylamidoether sulfates, alkylarylpolyether sulfates, monoglyceride sulfates, alpha-olefin sulfonates, paraffin sulfonates, alkyl phosphates, alkyl ether phosphates, alkyl sulfonates, alkylamide sulfonates, alkylaryl sulfonates, alkyl carboxylates, alkyl sulfosuccinates, alkyl ether sulfosuccinates, alkylamide sulfosuccinates, alkyl sulfoacetates, alkyl sarcosinates, acyl isethionates, N-acyl taurates, acyl lactylates, N-acyl derivatives of amino acids, N-acyl derivatives of peptides, N-acyl derivatives of proteins, or fatty acids.

“Cosmetically acceptable” amphoteric foaming and/or detergent surfactants include alkyl betaines, alkylamidobetaines, sultaines, alkylamidoalkylsulfobetaines, imidazoline derivatives, phosphobetaines, amphopolyacetates and amphopropionates.

“Cosmetically acceptable” cationic foaming and/or detergent surfactants include quaternary ammonium derivatives.

“Cosmetically acceptable” non-ionic foaming and/or detergent surfactants include alkyl polyglycosides comprising a linear or branched, saturated or unsaturated aliphatic radical comprising 8 to 12 carbon atoms, castor oil derivatives, polysorbates, cocamides and N-alkylamines.

“Cosmetically acceptable” thickening and/or gelling surfactants include in particular:
optionally alkoxylated alkylpolyglycoside fatty esters, such as ethoxylated methylpolyglucoside esters such as PEG 120 methyl glucose trioleate and PEG 120 methyl glucose dioleate, sold respectively under the names GLUCAMATE™ LT and GLUMATE™ DOE120;
alkoxylated fatty esters such as PEG 150 pentaerythrityl tetrastearate and PEG 55 propylene glycol oleate, sold respectively under the names CROTHIX™ DS53 and ANTIL™ 141;
fatty-chain polyalkylene glycol carbamates such as PPG 14 laureth isophoryl dicarbamate and PPG 14 palmeth 60 hexyl dicarbamate, sold respectively under the names ELFACOS™ T211 and ELFACOS™ GT2125.

“Cosmetically acceptable” emulsifying surfactants include in particular non-ionic surfactants, anionic surfactants and cationic surfactants.

Examples of “cosmetically acceptable” non-ionic emulsifying surfactants include sorbitol fatty acid esters, for example the products sold under the names MONTANE™80 and MONTANE™85 and MONTANE™60; ethoxylated castor oil and ethoxylated hydrogenated castor oil, for example the product sold under the name SIMULSOL™ 989; compositions comprising glycerol stearate and poly(ethoxylated) stearic acid having between 5 mol and 150 mol of ethylene oxide, for example the composition comprising (ethoxylated) stearic acid having 135 mol of ethylene oxide and glycerol stearate sold under the name SIMULSOL™ 165; ethoxylated sorbitan esters, for example the products sold under the name MONTANOX™—mannitan esters; ethoxylated mannitan esters; sucrose esters; methylglucoside esters.

Examples of “cosmetically acceptable” anionic emulsifying surfactants include decylphosphate, cetylphosphate sold under the name AMPHISOL™, glyceryl stearate citrate; cetearyl sulfate, the arachidyl/behenyl phosphates and arachidyl/behenyl alcohols composition sold under the name SENSANOV™ WR, sodium stearate or triethanolammonium stearate, N-acyl derivatives of salified amino acids, for example stearoyl glutamate.

Examples of “cosmetically acceptable” cationic emulsifying surfactants include amine oxides, quaternium-82 and the surfactants described in the international application published under number WO 96/00719, and predominantly those with a fatty chain comprising at least 16 carbon atoms.

Examples of “cosmetically acceptable” opacifying and/or pearlizing agents include sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate, and fatty alcohols comprising from 12 to 22 carbon atoms.

Examples of “cosmetically acceptable” texturizing agents include N-acyl derivatives of amino acids, for example lauroyl lysine, octenyl starch succinate and myristyl polyglucoside, sold respectively under the names AMINOHOPE™ LL, DRYFLO™ and MONTANOV 14; cellulose fibers, cotton fibers, chitosan fibers, talc, sericite, mica.

Examples of “cosmetically acceptable” solvents and cosolvents, alone or as mixtures, include water, glycerol, diglycerol, glycerol oligomers, ethylene glycol, propylene glycol, butylene glycol, hexylene glycol, diethylene glycol, xylitol, erythritol, sorbitol and water-soluble alcohols such as ethanol, isopropanol or butanol.

Examples of “cosmetically acceptable” deodorants include alkali metal silicates, zinc salts such as zinc sulfate, zinc gluconate, zinc chloride, zinc lactate; quaternary ammonium salts such as cetyltrimethylammonium salts, cetylpyridinium salts; glycerol derivatives such as glycerol caprate, glycerol caprylate, polyglycerol caprate; 1,2-decanediol; 1,3-propanediol; salicylic acid; sodium bicarbonate; cyclodextrins; metal zeolites; Triclosan™; aluminum bromohydrate, aluminum chlorohydrates, aluminum chloride, aluminum sulfate, aluminum and zirconium chlorohydrates, aluminum and zirconium trichlorohydrate, aluminum and zirconium tetrachlorohydrate, aluminum and zirconium pentachlorohydrate, aluminum and zirconium octochlorohydrate, aluminum sulfate, sodium and aluminum lactate, aluminum glycol chlorohydrate complexes, such as aluminum propylene glycol chlorohydrate complex, aluminum propylene glycol dichlorohydrate complex, aluminum propylene glycol sesquichlorohydrate complex, aluminum polyethylene glycol chlorohydrate complex, aluminum polyethylene glycol dichlorohydrate complex, aluminum polyethylene glycol sesquichlorohydrate complex.

Examples of “cosmetically acceptable” antioxidants include EDTA and the salts thereof, citric acid, tartaric acid, oxalic acid, BHA (butylated hydroxyanisole), BHT (butylated hydroxytoluene), tocopherol derivatives such as tocopherol acetate, mixtures of antioxidant compounds such as DISSOLVINE GL 47S (INCI name: Tetrasodium Glutamate Diacetate).

Examples of active ingredients optionally present in the emulsions (E), (E₁) and (E₂) as previously defined include:
vitamins and the derivatives thereof, in particular the esters thereof, such as retinol (vitamin A) and the esters thereof (for example retinyl palmitate), ascorbic acid (vitamin C) and the esters thereof, sugar derivatives of ascorbic acid (for example ascorbyl glucoside), tocopherol (vitamin E) and the esters thereof (e.g. tocopherol acetate), vitamins B3 or B10 (niacinamide and the derivatives thereof);
compounds exhibiting a soothing action, in particular SEPICALM™ S, allantoin and bisabolol;
compounds exhibiting a moisturizing action, such as urea, hydroxyureas, glyceryl glucoside, diglyceryl glucoside, polyglyceryl glucosides, glycerol, diglycerol, xylityl polyglucoside sold under the trade name Aquaxyl™;
polyphenol-rich plant extracts such as grape extracts, pine extracts, wine extracts, olive extracts, N-acyl peptides such as MATRIXIL™; N-acyl amino acids; N-acyl partial protein hydrolysates; amino acids; peptides; total protein hydrolysates; soy extracts, for example Raffermine™; wheat extracts, for example TENSINE™ or GLIADINE™; tannin-rich plant extracts, isoflavone-rich plant extracts or terpene-rich plant extracts;
freshwater or marine algae extracts; marine plant extracts; marine extracts in general, such as coral; essential waxes; bacterial extracts;
ceramides; phospholipids; compounds exhibiting antimicrobial or purifying action, such as LIPACIDE™ C8G, LIPACIDE™ UG, SEPICONTROL™ A5; OCTOPIROX™ or SENSIVA™ SC50;
compounds exhibiting energizing or stimulating properties such as PHYSIOGENYL™, panthenol and the derivatives thereof, such as SEPICAP™ MP; anti-aging active ingredients such as SEPILIFT™ DPHP, LIPACIDE™ PVB, SEPIVINOL™, SEPIVITAL™, MANOLIVA™, PHYTO-AGE™, TIMECODE™, SURVICODE™;
anti-photoaging active ingredients; active ingredients that protect the integrity of the dermal-epidermal junction;
active ingredients that increase the synthesis of extracellular matrix components such as collagen, elastins and glycosaminoglycans;
active ingredients that have a positive effect on chemical cell communication, such as cytokines, or physical cell communication, such as integrins;
active ingredients that create a “warming” sensation on the skin, such as skin microcirculation activators (such as nicotinic acid derivatives) or products that create a “cooling” sensation on the skin (such as menthol and derivatives thereof);
active ingredients that improve skin microcirculation, such as venotonic agents; draining active ingredients; decongesting active ingredients such as extracts of Ginkgo biloba, ivy, horse chestnut, bamboo, ruscus, butcher's broom, Centella asiatica, fucus, rosemary and willow.

The emulsions (E), (E₁) and (E₂) which are the subject of the present invention can be prepared by a method known to those skilled in the art which comprises in particular the following steps:
a step of preparing said aqueous phase (A) by mixing all the hydrophilic components in water;
a step of preparing said fatty phase (G) by mixing all the hydrophilic lipophilic components in the oil or mixture of oils and/or wax constituting said phase (G), then adding said composition (C) as defined above thereto;
a step of mixing, at the appropriate temperature, said aqueous phase (A) and said fatty phase (G), with mechanical stirring using an anchor-type spindle and at a speed of 60 rpm to a speed of approximately 100 rpm, until a homogeneous emulsion is obtained;—optionally, a step of adjusting the pH of the emulsion.

The emulsions (E), (E₁) and (E₂) can be packaged in pressurized form, for example in an aerosol device, in a “pump bottle” type device or in a device provided with a perforated wall, for example a mesh.

According to another more particular aspect, the invention relates to the use as defined previously, characterized in that said composition (C) is implemented in a cosmetic formulation (F) comprising from 1% to 10% by weight thereof and more particularly from 2% to 5% by weight, for 100% of the weight thereof.

Another subject of the invention is a method for drying the hair, characterized in that it comprises the following consecutive steps:
at least one step a) of applying to the hair a cosmetic formulation (F) comprising, for 100% of the weight thereof, from 0.2% to 40% by weight of a composition (C), said composition (C) comprising, for 100% of the weight thereof,
from 10% by weight to 90% by weight of at least one poly(farnesene) polymer having a number-average molecular weight of greater than or equal to 10,000 g/mol and less than or equal to 120,000 g/mol, said poly(farnesene) polymer being partially or totally hydrogenated and/or functionalized with hydroxyl radicals, and
from 10% by weight to 90% by weight of a mixture (M) of hydrocarbons in which at least 94% by weight of said hydrocarbons comprise from fifteen to nineteen carbon atoms,—optionally, a step b) during which, at the end of step a), the hair is left to rest at room temperature for a duration of 5 to 60 minutes, then
a step c) of drying the hair at the end of step a) or optionally at the end of step b), by means of a heating appliance such as a hair dryer or heated hood.

In step a) of the drying method as defined above, said formulation (F) is applied directly or indirectly, for example by means of any type of support intended to be brought into contact with the skin or hair, such as paper, wipes or textiles.

In step b of the drying method as defined above, “room temperature” means a temperature of between 15° C. and 35° C., in particular between 20° C. and 30° C.

According to a particular aspect of the method as defined above, said mixture (M) of hydrocarbons present in the composition (C) contained in the formulation (F) implemented in step a) is a mixture of saturated hydrocarbons comprising, for 100% of the weight thereof:
from 15% to 20% by weight of alkanes (linear, isoalkanes and cycloalkanes) comprising 17 carbon atoms,
from 70% to 75% by weight of alkanes (linear, isoalkanes and cycloalkanes) comprising 18 carbon atoms, and
from 4% to 6% by weight of alkanes (linear, isoalkanes and cycloalkanes) comprising 19 carbon atoms.

According to a more particular aspect of the method as defined above, said mixture (M) of hydrocarbons present in the composition (C) contained in the formulation (F) implemented in step a) is a mixture of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms. According to another particular aspect of the method as defined above, said poly(farnesene) polymer present in the composition (C) contained in the formulation (F) implemented in step a) has a number-average molecular weight of greater than or equal to 20,000 g/mol and less than or equal to 90,000 g/mol, and in particular a number-average molecular weight of greater than or equal to 40,000 g/mol and less than or equal to 80,000 g/mol; said poly(farnesene) polymer present in said composition (C) contained in the formulation (F) implemented in step a) is more particularly totally hydrogenated, functionalized with hydroxyl radicals and has a number-average molecular weight equal to 71,000 g/mol.

According to a most particular aspect of the method as defined above, said composition (C) contained in the formulation (F) implemented in step a) comprises, for 100% of the weight thereof,
40% by weight of a totally hydrogenated poly(farnesene) polymer functionalized with hydroxyl groups and having a number-average molecular weight equal to 71,000, and
60% by weight of a mixture (M) of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms.

According to another particular aspect of the method as defined above, said composition (C) is implemented in an emulsion (E) of an aqueous phase (A) and a fatty phase (G) comprising, for 100% of the weight thereof:
from 50% to 98% by weight of said aqueous phase (A), and
from 2% to 50% by weight of said fatty phase (G),
said fatty phase (G) comprising, for 100% of the weight thereof:
from 10% to 80% by weight of said fatty composition (C),
from 0.5% to 20% by weight of an emulsifying surfactant (S) or a mixture of emulsifying surfactants (Ms) of the oil-in-water or water-in-oil type, and
from 0% to 89.5% by weight of an oil or a plurality of oils and/or a wax, said wax being different from said composition (C).

According to another particular aspect of the method as defined above, said composition (C) is implemented in a cosmetic formulation (F) comprising, for 100% of the weight thereof, from 1% to 10% thereof, and more particularly from 2% to 5% by weight thereof.

Another subject of the invention is a method for shaping the hair, characterized in that it comprises the following consecutive steps:
a step d) of applying, to dry or wet hair, a cosmetic formulation (F) comprising, for 100% of the weight thereof, 0.2% to 40% by weight of a composition (C) comprising, for 100% of the weight thereof,
from 10% by weight to 90% by weight of at least one poly(farnesene) polymer having a number-average molecular weight of greater than or equal to 10,000 g/mol and less than or equal to 120,000 g/mol, said poly(farnesene) polymer being partially or totally hydrogenated and/or functionalized with hydroxyl radicals, and
from 10% by weight to 90% by weight of a mixture (M) of hydrocarbons in which at least 94% by weight of said hydrocarbons comprise from fifteen to nineteen carbon atoms,
optionally a step e) during which, at the end of step d), the hair is left to rest at room temperature for a duration of 5 to 60 minutes, then,
a step f) of shaping the hair at the end of step d) or optionally at the end of step e), by means of a heating appliance such as a straightening iron or curling tongs.

In the definition of the method as defined above, “hair shaping” means the actions of combing, straightening, waving and/or curling the hair.

In the shaping method as defined above, “room temperature” means a temperature of between 15° C. and 35′C, in particular between 20° C. and 30° C.

In step d) of the shaping method as defined above, said formulation (F) is applied directly or indirectly, for example by means of any type of support intended to be brought into contact with the skin or hair, such as paper, wipes or textiles.

According to a more particular aspect of the method as defined above, said mixture (M) of hydrocarbons present in the composition (C) contained in the formulation (F) implemented in step a) is a mixture of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms. According to another particular aspect of the method as defined above, said poly(farnesene) polymer present in the composition (C) contained in the formulation (F) implemented in step a) has a number-average molecular weight of greater than or equal to 20,000 g/mol and less than or equal to 90,000 g/mol, and in particular a number-average molecular weight of greater than or equal to 40,000 g/mol and less than or equal to 80,000 g/mol; said poly(farnesene) polymer present in said composition (C) contained in the formulation (F) implemented in step a is more particularly totally hydrogenated, functionalized with hydroxyl radicals and has a number-average molecular weight equal to 71,000 g/mol.

According to a most particular aspect of the method as defined above, said composition (C) contained in the formulation (F) implemented in step a) comprises, for 100% of the weight thereof,
40% by weight of a totally hydrogenated poly(farnesene) polymer functionalized with hydroxyl groups and having a number-average molecular weight equal to 71,000, and
60% by weight of a mixture (M) of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms.

According to another particular aspect of the method as defined above, said composition (C) is implemented in an emulsion (E) of an aqueous phase (A) and a fatty phase (G) comprising, for 100% of the weight thereof:
from 50% to 98% by weight of said aqueous phase (A), and
from 2% to 50% by weight of said fatty phase (G), said fatty phase (G) comprising, for 100% of the weight thereof:
from 10% to 80% by weight of said fatty composition (C),
from 0.5% to 20% by weight of an emulsifying surfactant (S) or a mixture of emulsifying surfactants (Ms) of the oil-in-water or water-in-oil type, and
from 0% to 89.5% by weight of an oil or a plurality of oils and/or a wax, said wax being different from said composition (C).

According to another particular aspect of the method as defined above, said composition (C) is implemented in a cosmetic formulation (F) comprising, for 100% of the weight thereof, from 1% to 10% thereof, and more particularly from 2% to 5% by weight thereof.

### EXPERIMENTAL EXAMPLES

### Preparation of the Composition (C) Implemented in the Examples

The (poly)farnesene polymer contained in composition (C) implemented in the following examples is totally hydrogenated and functionalized with hydroxyl radicals; it has a number-average molecular weight of 71,000 g/mol. It was prepared according to the procedure described in WO2018/052709.

The mixture (M) contained in this same composition (C) is commercially available under the trade name Emogreen™ L19; it comprises, for 100% of the weight thereof, 3.8% linear alkanes comprising from fifteen to nineteen carbon atoms, 96.2% isoalkanes containing from fifteen to nineteen carbon atoms, and a proportion by weight of less than 0.1% of cycloalkanes comprising from fifteen to nineteen carbon atoms; of these constituents, for 100% of the weight thereof, 17.1% by weight are alkanes (linear, isoalkanes and cycloalkanes) comprising 17 carbon atoms, 72.5% by weight are alkanes (linear, isoalkanes and cycloalkanes) comprising 18 carbon atoms, and 4.7% by weight are alkanes (linear, isoalkanes and cycloalkanes) comprising 19 carbon atoms. The mixture (M) was prepared according to the procedure described in WO 2020/144440 A1.

The desired amount of Emogreen™ L19 is poured into a glass reactor equipped with a double jacket in which a temperature-controlled fluid circulates, and equipped with a mechanical stirrer fitted with an anchor-type paddle. The stirring speed is set to 50 rpm and the temperature to 25° C. The desired amount of poly(farnesene) polymer is then introduced with moderate stirring at 60° C. The amounts introduced are such that Emogreen™ L19 represents a content by weight of 60% and the poly(farnesene) polymer represents a content by weight of 40%, for 100% by weight of the composition (C) thus obtained.

Demonstration of the compatibility of composition (C) in formulations intended to be used by application to the hair.

### A. Compatibility of Composition (C) in Hair Conditioning Formulations Comprising a Cationic Conditioning Agent.

Preparation of the formulations: The formulations according to the invention (F₁) and (F₂) and the comparative formulations (F′₁) and (F′₂) are prepared, the compositions of which are given in the following Table 1, the proportions being expressed as percentages by weight.

Evaluation of the formulations: A 100 cm³ amount of the composition to be tested, contained in a 250 cm³ bottle, is introduced into a climatic chamber regulated to 20° C., for a period of 3 months. The visual appearance of the composition being tested is evaluated after a period of 7 days and after a period of 3 months. The same operation is repeated with new samples of the formulations, regulating the temperature inside the climatic chamber to 45° C. The visual appearance of the composition being tested is evaluated after a period of 3 months. The results obtained are set out in Table 2 below.

TABLE 1

| | Proportions by weight of the constituents | | | |
|---|---|---|---|---|
| | for each of the formulations | | | |
| | (F₁) | (F₂) | (F′₁) | (F′₂) |
| Water | q.s. to | q.s. to | q.s. to | q.s. to |
| | 100% | 100% | 100% | 100% |
| Dissolvine ™ GE47 | 0.30% | 0.30% | 0.30% | 0.30% |
| Glycerol | 3.00% | 3.00% | 3.00% | 3.00% |
| AG C₁₆-C₁₈ | 4.00% | 4.00% | 4.00% | 4.00% |
| Varisoft ™ BT85 | 3.00% | 3.00% | 3.00% | 3.00% |
| DE alpha-tocopherol | 0.05% | 0.05% | 0.05% | 0.05% |
| Coconut oil | 2.00% | 2.00% | 2.00% | 2.00% |
| Eanol ™ 2681 | 3.00% | 3.00% | 3.00% | 3.00% |
| Natrosol ™250HPC | 0.80% | 0.80% | 0.80% | 0.80% |
| Composition (C) | 3.00% | 5.00% | 0% | 0% |
| Massocare ™ SIE CPD | 0% | 0% | 3.00% | 5.00% |
| Euxyl ™ PE9010 | 1.00% | 1.00% | 1.00% | 1.00% |
| Citric acid | q.s. to | q.s. to | q.s. to | q.s. to |
| | pH 4.5 | pH 4.5 | pH 4.5 | pH 4.5 |

TABLE 2

| | (F₁) | (F₂) | (F′₁) | (F′₂) |
|---|---|---|---|---|
| | Appearance of the formulations | | | |
| After 7 days at 20° C. | Homogeneous | Homogeneous | Homogeneous | Homogeneous |
| After 3 months at 20° C. | Homogeneous | Homogeneous | Homogeneous | Homogeneous |
| After 3 months at 45° C. | Homogeneous | Homogeneous | Exudation | Exudation |
| | Viscosity at 20° C. (BKF RVT M4V6) | | | |
| After 7 days at 20° C. | 62,000 mPa · s | 76,000 mPa · s | 66,000 mPa · s | 78,000 mPa · s |
| After 3 months at 20° C. | 68,000 mPa · s | 77,000 mPa · s | 70,000 mPa · s | 68,000 mPa · s |

### B. Compatibility of a Composition (C) in Hair Conditioning Emulsions of Oil-In-Water Type Comprising a Crosslinked Cationic Polyelectrolyte.

Preparation of the formulations: The formulations (F₃) and (F₄) according to the invention and the comparative formulations (F′₃) and (F′₄) are prepared, the compositions of which are given in the following Table 3, the proportions being expressed as percentages by weight.

Evaluation of the formulations: The formulations (F₃), (F₄), (F′₃) and (F′₄) are evaluated using the same protocol as described in “Evaluation of the formulations” in paragraph A above. The results obtained are set out in Table 4 below.

TABLE 3

| | Proportions by weight of the constituents for each of | | | |
|---|---|---|---|---|
| | the formulations | | | |
| | (F₃) | (F₄) | (F′₃) | (F′₄) |
| Water | q.s. to 100% | q.s. to 100% | q.s. to 100% | q.s. to 100% |
| SOLAGUM ™ Tara | 0.30% | 0.30% | 0.30% | 0.30% |
| Glycerol | 2.00% | 2.00% | 2.00% | 2.00% |
| Argan oil | 2.00% | 2.00% | 2.00% | 2.00% |
| DL alpha-tocopherol | 0.50% | 0.50% | 0.50% | 0.50% |
| Simulquat ™ HC305 | 3.00% | 3.00% | 3.00% | 3.00% |
| Composition (C) | 3.00% | 5.00% | 0% | 0% |
| Massocare ™ SIL | 0% | 0% | 3.00% | 5.00% |
| CPD | | | | |
| Euxyl PE9010 | 1.00% | 1.00% | 1.00% | 1.00% |
| Citric acid | q.s. to pH 4.5 | q.s. to pH 4.5 | q.s. to pH 4.5 | q.s. to pH 4.5 |

TABLE 4

| | (F₃) | (F₄) | (F′₃) | (F′₄) |
|---|---|---|---|---|
| | Appearance of the formulations | | | |
| Appearance after 7 | Homogeneous | Homogeneous | Homogeneous | Homogeneous |
| days at 20° C. | | | | |
| Appearance after 3 | Homogeneous | Homogeneous | Homogeneous | Homogeneous |
| months at 20° C. | | | | |
| After 3 months at | Homogeneous | Homogeneous | Homogeneous | Exudation |
| 45° C. | | | | |
| | Viscosity at 20° C. (BKF RVT M4V6) | | | |
| After 7 days at | 167,000 mPa · s | 105,000 mPa · s | 180,000 mPa · s | 104,000 mPa · s |
| 20° C. | | | | |
| After 3 months at | 157,000 mPa · s | 95,000 mPa · s | 157,000 mPa · s | 75,000 mPa · s |
| 20° C. | | | | |

### C. Compatibility of a Composition (C) in Unrinsed Hair Conditioning Emulsions of Oil-In-Water Type Comprising at Least One Surfactant and Intended to be Applied to the Hair.

Preparation of the formulations: The formulations (F₅) and (F₆) according to the invention and the comparative formulations (F′₅) and (F′₆) are prepared, the compositions of which are given in the following Table 5, the proportions being expressed as percentages by weight.

TABLE 5

| | Proportions by weight of the constituents for each of the | | | |
|---|---|---|---|---|
| | formulations | | | |
| | (F₅) | (F₆) | (F′₅) | (F′₆) |
| Water | q.s. to 100% | q.s. to 100% | q.s. to 100% | q.s. to 100% |
| Dissolvine GL47 | 0.30% | 0.30% | 0.30% | 0.3% |
| Sepimax ™ Zen) | 0.60% | 0.60% | 0.60% | 0.60% |
| Fluidifoel ™ Easy | 3.00% | 3.00% | 3.00% | 3.00% |
| Argan oil | 3.00% | 2.00% | 3.00% | 2.00% |
| DL alpha-tocopherol | 0.050% | 0.05% | 0.05% | 0.05% |
| Composition (C) | 2.00% | 3.00% | 0% | 0% |
| Massocare ™ SIL CPD | 0% | 0% | 2.00% | 3.00% |
| Euxyl ™ PE9010 | 1.00% | 1.00% | 1.00% | 1.00% |
| Citric acid | q.s. to pH 4.5 | q.s. to pH 4.5 | q.s. to pH 4.5 | q.s. to pH 4.5 |

Evaluation of the formulations: The formulations (F₅), (F₆), (F′₅) and (F′₆) are evaluated using the same protocol as above. The results are set out in Table 6 below.

TABLE 6

| | (F₅) | (F₆) | (F′₅) | (F′₆) |
|---|---|---|---|---|
| | Appearance of the formulations | | | |
| After 7 days at | Homogeneous, | Homogeneous, | Heterogeneous, | Heterogeneous, |
| 20° C. | smooth liquid | smooth liquid | smooth liquid | smooth liquid |
| | | | with globules | with globules |
| After 3 months at | Homogeneous, | Homogeneous, | Heterogeneous, | Heterogeneous, |
| 20° C. | smooth liquid | smooth liquid | smooth liquid | smooth liquid |
| | | | with globules | with globules |
| After 3 months at | Homogeneous, | Homogeneous, | Heterogeneous, | Heterogeneous, |
| 45° C. | smooth liquid | smooth liquid | smooth liquid | smooth liquid |
| | | | with globules | with globules |
| | Viscosity at 20° C. (BKF RVT M2V6): | | | |
| After 7 days at | 2440 mPa · s | 2620 mPa · s | 1540 mPa · s | 2480 mPa · s |
| 20° C. | | | | |
| After 3 months at | 2420 mPa · s | 2155 mPa · s | 1720 mPa · s | 2200 mPa · s |
| 20° C. | | | | |

### D. Compatibility of a Composition (C) in Oils for Application to the Hair

Preparation of the formulations: The formulation according to the invention (F₇) and the comparative formulations (F′₇) and (F′₈) are prepared, the compositions of which are given in the following Table 7, the proportions being expressed as percentages by weight.

TABLE 7

| | Proportions by weight of the constituents | | |
|---|---|---|---|
| | for each of the formulations | | |
| | (F₇) | (F′₈) | (F′₇) |
| Composition (C) | 50% | / | / |
| Emogreen L19 | / | / | 50% |
| Massocare SIL CPD | / | 50% | / |
| Argan oil | 2.50% | 2.50% | 2.50% |
| Coconut oil | 2.50% | 2.50% | 2.50% |
| Triglycerides 5545 | 44.80% | 44.80% | 44.80% |
| DL alpha-tocopherol | 0.20% | 0.20% | 0.20% |

Evaluation of the formulations: The formulations (F₇), (F′₇), and (F′₈) are evaluated using the same protocol as above. The results are set out in Table 8 below.

TABLE 8

| | (F₇) | (F′₈) | (F′₇) |
|---|---|---|---|
| | Appearance of the formulations | | |
| After 7 days at 20° C. | Homogeneous | Heterogeneous, | Homogeneous |
| | | immiscible | |
| After 3 months at | Homogeneous | Heterogeneous, | Homogeneous |
| 20° C. | | immiscible | |
| After 3 months at | Homogeneous | Heterogeneous, | Homogeneous |
| 45° C. | | immiscible | |
| | Viscosity at 20° C. (BKF LVT M2): | | |
| | 120 mPa · s | Not measurable | <50 mPa · s |

### E. Observations and Conclusions

The results obtained in the previous paragraphs lead to the following conclusions:
The formulations (F₁) and (F₂) according to the invention comprising a conditioning agent and composition (C) exhibit a homogeneous appearance after storage for a period of 3 months at 45′C, which is not observed for the formulations (F′₁) and (F′₂) comprising silicone derivatives.
The emulsion of W/O type (formulation (F₄)) according to the invention comprising a crosslinked cationic polyelectrolyte and 5% of composition (C) exhibits a homogeneous appearance after storage for a period of 3 months at 45° C., whereas the formulation (F′₄) comprising silicone derivatives exudes.
Unrinsed hair conditioning emulsions of O/W type comprising at least one surfactant and composition (C) have a more homogeneous appearance than the comparative emulsions (formulations (F′₅ and F′₆)) which comprise globules.
The formulation (F₇) comprising the composition (C) has a homogeneous appearance after storage for 3 months at 45° C., which is not observed with the formulation (F's), which has a heterogeneous appearance.

We can therefore conclude from all these results that formulations comprising the composition (C) are highly compatible with formulation schemes intended for applications in hair care and/or hair hygiene.

Demonstrating the preservation of keratin integrity in the hair cortex of a formulation comprising composition (C).

The effect of said composition (C) in preserving the integrity of keratin in the hair cortex is evaluated using the Xpolar® technology developed by Kmax Innovative System. A.

### A. Principle of the Evaluation Method

Xpolar® technology is an integrated imaging mode on a microscopy platform, enabling the polarimetric changes produced by the samples being studied to be observed and measured. It provides information about the birefringence of the sample being evaluated. “Birefringence” means the physical property of a material in which light propagates anisotropically. In a birefringent medium, the refractive index is not unique; it depends on the direction of polarization of the light wave. The “k-index” parameter measured by Xpolar® technology provides information on the birefringence of the sample at each point in the image, and can be used quantitatively to characterize the sample. Hair is composed of keratins, a family of fibrous proteins. Keratin fibers comprise a crystalline part bonded by Coulomb forces and hydrophobic interactions. This crystalline structure of the keratin fibers in the cortex gives hair the ability to modify the polarization of light passing through it, through a property referred to as birefringence. Healthy (control) hair, i.e. hair that has not been exposed to intense heat, has a high k-index value. The color within the fiber is homogeneous and is composed of the colors at the maximum level of the measurement (dominant red-orange coloring). When the keratin fibers within the hair are not adversely affected, in particular by exposure to intense heat, they are organized in a crystalline network, resulting in a “high k-index” value. In contrast, exposure of the hair to thermal stress, such as exposure to intense heat, modifies the internal structure of the keratin, leading to disorganization and a change in the optical properties of the keratin, ultimately resulting in a reduction in the “k-index” and a sharp decrease in color within the fiber, which becomes heterogeneous and shows different hues (green, blue, purple).

### B. Procedure of the Evaluation Method

The composition to be tested is applied to dry hair with a comb comprising approximately 30 mg of said composition to be tested. The hair is then massaged for 30 seconds, then dried at room temperature (20° C.) for 30 minutes. The heat treatment is then carried out with a straightening iron at a temperature of 210° C. for 50 passes. The hair treated in this way is left at room temperature for 30 minutes.

Observation slides are prepared by embedding the hair in a freezing medium, then freezing (−20° C.) the embedded hair, then making a 30 μm cut and mounting the slides on the observation apparatus (Xpolar apparatus, magnification: ×20 and acquisition of 25 images per condition).

### C. Evaluated Compositions

Three compositions and four formulas, identified in Tables 9 and 10, respectively, were evaluated.

TABLE 9

| References | LCE21076 | LCE21077 | LCE21078 |
|---|---|---|---|
| Ingredient | Composition (C) | Massocare ™ SIL | Emogreen ™ L19 |
| | | DMD | |
| Appearance | Colorless liquid | Colorless liquid | Colorless liquid |

TABLE 10

| References | LCE21071 | LCE21072 | LCE21073 | LCE21075 |
|---|---|---|---|---|
| Demineralized water | q.s. to 100% | | | |
| Dissolvine ™ GL47 | 0.30% | | | |
| Sepimax ™ Zen | 0.60% | | | |
| Fluidifeel ™ Easy | 3.00% | | | |
| Argan oil | 2.00% | | | |
| DL alpha-tocopherol | 0.05% | | | |
| Composition (C1) | 3.00% | 0% | 0% | 0% |
| Massocare ™ SIL DMD | 0% | 3.00% | 0% | 0% |
| Emogreen ™ L19 | 0% | 0% | 0% | 3.00% |

| | |
|---|---|
| Euxyl ™ PE9010 | 1.00% |
| Lactic acid | q.s. to pH = 4.5-5.0 |

In addition, the same evaluation was carried out with untreated hair that had not undergone heat treatment (‘negative control’), and with hair that was untreated but had undergone the heat treatment described in the above procedure (‘positive control’).

### D. Results Obtained

The “k-index” values obtained are set out in Table 11 below. The results are expressed by two values:

The variation with respect to the negative control VA₁, calculated as follows: VA₁=[(k-indexᵢ;−k-index_{negative control})×100/k-index_{negative control}
The percentage protection VA₂, calculated as follows: VA₂=100−[(k-indexᵢ;−k-index_{negative control})×100/(k-index_{negative control} k-index_{positive control})]

TABLE 11

| Composition tested | k-index (×10⁴) | VA₁ | VA₂ |
|---|---|---|---|
| Negative control | 87.82 ± 16.13 | | |
| Positive control | 58.42 ± 9.70 | −33% | |
| LCE 21076 | 69.65 ± 9.70 | −21% | 38.2% |
| LCE 21077 | 68.16 ± 13.45 | −22% | 33.1% |
| LCE 21078 | 51.83 ± 6.56 | −41% | −22.4% |
| LCE 21071 | 60.64 ± 11.65 | −31% | 22.0% |
| LCE 21072 | 67.24 ± 11.81 | −23% | 40.9% |
| LCE 21073 | 52.96 ± 8.11 | −40% | na |
| LCE 21075 | 62.35 ± 8.88 | −29% | 26.9% |

An additional statistical analysis was carried out between the different compositions, showing the significant differences expressed in Table 12 below:

TABLE 12

| Studied conditions | Significant difference (p) |
|---|---|
| LCE 21073/LCE 21071 | Yes (p = 9 10⁻³) |
| LCE 21073/LCE 21072 | Yes (p = 3.5 10⁻⁵) |
| LCE 21073/LCE 21075 | Yes (p = 2.8 10⁻⁴) |
| LCE 21071/LCE 21072 | No (p = 8 10⁻²) |
| LCE 21076/LCE 21077 | No (p = 0.72) |
| LCE 21076/LCE 21078 | Yes (p = 1.14 10⁻⁵) |

Observations and comments: Composition (C) (LCE21076) and composition (LCE21077) based on silicone derivatives show a percentage protection against exposure to the heat of the straightening iron of 38.2% and 33.1% respectively; composition (LCE21078) based on the hydrocarbon mixture Emogreen™ L19 shows a negative percentage protection, thus demonstrating its ineffectiveness, and visual observation during experimentation highlighted the strongly degraded condition of the keratin. Among the formulations tested, for the “placebo” formulation (LCE21073), so-called because it does not comprise composition (C), silicone derivatives or any other film-forming agents or keratin-protecting agents, keratin damage caused by heat treatment was observed. The percentage protection results show that the 3 formulations (LCE21071) (LCE21072) (LCE21075) provide keratin protection that is significantly different to that provided by the placebo formula (LCE21073). Composition (C) shows a protective action on keratin in the hair cortex, reducing the effect of heat on keratin integrity by 38%. This property was verified in a formulation versus placebo, where the keratin protection rate was 22%. Formulations according to the invention comprising composition (C) are therefore alternative solutions to silicone derivatives, for example polydimethylsiloxane (PDMS or dimethicone), and/or dimethiconols, for providing cosmetic formulations with properties that make it possible to slow down or prevent the appearance of unattractive effects on human hair following exposure to intense heat. This alternative implements chemical compositions of plant and/or biodegradable origin.

### Examples of Cosmetic Formulations

Water-in-oil emulsion for hair care before drying

| Composition of the emulsion | Proportions by weight of constituents |
|---|---|
| Water | q.s. to 100% |
| Glycerol | 2.00% |
| Simulquat ™ HC 305(40% active | 2.50% (i.e. 1% active substance) |
| substance) | |
| Triethanolamine 6% | Up to pH = 4.0-4.5 |
| Easynov ™ | 2.50% |
| Composition (C) | 10.00% |
| Euxyl ™ PE 9010 | 1.00% |

Hair balm of oil-in-water emulsion type

| Composition of the balm | Proportions by weight of constituents |
|---|---|
| Demineralized water | q.s. to |
| Dissolvine ™ GL47S | 0.30% |
| Sepimax ™ Zen | 0.60% |
| Solagum ™ AX | 0.50% |
| Fluidifeel ™ Easy | 0.30% |
| Argan oil | 2.00% |
| DL alpha-tocopherol | 0.05% |
| Composition (C) | 3.00% |
| Euxyl ™ PE9010 | 1.00% |
| Lactic acid | Up to pH = 4.5 |

Sublime hair mask

| | Proportions by weight |
|---|---|
| Composition of the mask | of constituents |
| Aqua/Water | q.s. to 100% |
| Glycerol | 2.00% |
| SOLAGUM ™ TARA | 0.30% |
| Composition (C) | 3.00% |
| Cocos Nucifera Oil | 1.00% |
| Moringa Oleifera Seed Oil | 1.00% |
| SIMULQUAT ™ HC 305 | 3.50% |
| XYLISHINE ™ | 3.00% |
| fragrance | 0.20% |
| Phenoxyethanol - Ethylhexylglycerin | 1.00% |
| Dye | 0.08% |

Precious hair oil

| | Proportions by weight |
|---|---|
| Composition of the oil | of constituents |
| LANOL ™ 2681 | 48.60% |
| Composition (C) | 5.00% |
| Limnanthes Alba (Meadowfoam) Seed | 5.00% |
| Oil | |
| Cocos Nucifera Oil | 20.00% |
| Simmondsia Shinensis Seed Oil | 20.00% |
| Fragrance | 0.20% |
| Tocopherol | 0.20% |
| SEA SATIN ™ | 1.00% |

“Perfecting” smoothing serum for protecting
hair from heat, softness and suppleness

| | Proportions by weight |
|---|---|
| Composition of the serum | of constituents |
| Aqua/Water | q.s. to 100% |
| Tetrasodium Glutamate Diacetate | 0.30% |
| SEPIMAX ™ ZEN | 0.60% |
| SOLAGUM ™ TARA | 0.50% |
| FLUIDIFEEL ™ EASY | 3.00% |
| Composition (C) | 3.00% |
| Argania Spinosa Kernel Oil | 2.00% |
| Isopropyl myristate | 1.00% |
| ALARIANE ™ | 1.00% |
| XYLISHINE ™ | 3.00% |
| Tocopherol | 0.05% |
| fragrance | 0.20% |
| Phenoxyethanol - Ethylhexylglycerin | 1.00% |
| Lactic acid | 0.25% |

The compounds identified by their trade names in the various formulations and in the experimental example are explained below: Varisoft™ BT85 is an antistatic agent and conditioner, identified under the INCI name: Behentrimonium Chloride;)

AG C16-C18 is a mixture of cetyl and stearyl alcohols (weight ratio=50/50); Natrosol™ 250HPC is a trade name for hydroxyethylcellulose;
Massocare™ SIL CPD is an emollient, identified by the INCI name: Cyclopentasiloxane (and) PEG/PPG-18/18 Dimethicone);
SIMULQUAT™ HC305 is an emulsions stabilizer and thickener identified under the INCI name:
Acrylamidopropyltnmonium Chloride/Acrylates Copolymer (and) Isohexadecane (and) Coceth-7);
Easynov™ is an emulsifier of water-in-oil type, identified under the INCI name: octyldodecanol octyldodecyl xyloside and peg30 dipolyhydroxystearate);
Euxyl™ PE9010 is a preservative identified under the INCI name: phenoxyethanol and ethylhexylglycerin);
Dissolvine™ GL47 is a sequestrant, identified under the INCI name: Tetrasodium Glutamate Diacetate);
SEPIMAX™ Zen is a polymeric thickener used as a rheology modifier for cosmetic formulations and the polymeric backbone of which consists of monomeric units derived from 2-methyl 2-[(1-oxo-2-propenyl)amino]-1-propanesulfonic acid in sodium form, N,N-dimethylacrylamide, tetraethoxylated lauryl methacrylate and comprises trimethylolpropane triacrylate as crosslinking agent; it is identified under the INCI name: Polyacrylate Crosspolymer-6;
SOLAGUM™ TARA is a tara gum used as a texturizing agent and emulsion stabilizer, identified under the INCI name: Caesalpinia Spinosa Gum;
Fluidifeel™ Easy is a mixture of lauryl polyglucoside having a degree of polymerization of 1.20, myristyl polyglucoside having a degree of polymerization of 1.20, and polyglyceryl-6 laurate, used as an emulsifier of oil-in-water type to prepare cosmetic emulsions.

Xylishine™ is an active agent intended to improve hair glossiness, identified under the INCI name: Xylitylglucoside (and) Anhydroxylitol (and) Maltitol (and) Xylitol (and) Pelvetia Canaliculata Extract);

LANOL™ 2681 is a fatty phase with emollient properties, used for the preparation of cosmetic compositions identified under the INCI name: Coco-Caprylate/Caprate;
SEA SATIN™ is an oily extract of the marine plant Beta maritima, used in cosmetic compositions for its properties of enhancing volume, glossiness and suppleness of the hair; it is identified under the INCI name: Caprylic/Capric Triglyceride (and) Beta Vulgaris (Beet) Root Extract); Alariane™ is an ingredient used for its protective effect on hair exposed to external stresses (pollution, UV radiation); it is identified under the INCI name: Aqua (and) Butylene Glycol (and) Alaria Esculenta Extract.

1. A method for preventing and/or slowing the appearance of unattractive effects on human hair following exposure to intense heat, comprising applying to human hair a composition (C) comprising, for 100% of the weight thereof,
from 10% by weight to 90% by weight of at least one poly(farnesene) polymer having a number-average molecular weight of greater than or equal to 10,000 g/mol and less than or equal to 120,000 g/mol, said poly(farnesene) polymer being partially or totally hydrogenated and/or functionalized with hydroxyl radicals, and
from 10% by weight to 90% by weight of a mixture (M) of hydrocarbons in which at least 94% by weight of said hydrocarbons comprise from fifteen to nineteen carbon atoms.

2. The method according to claim 1, wherein said mixture (M) of hydrocarbons contained in said composition (C) is a mixture of saturated hydrocarbons comprising, for 100% of the weight thereof:
from 15% to 20% by weight of alkanes (linear, isoalkanes and cycloalkanes) comprising 17 carbon atoms,
from 70% to 75% by weight of alkanes (linear, isoalkanes and cycloalkanes) comprising 18 carbon atoms, and
from 4% to 6% by weight of alkanes (linear, isoalkanes and cycloalkanes) comprising 19 carbon atoms.

3. The method according to claim 2, wherein said mixture (M) of hydrocarbons present in said composition (C) is a mixture of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms.

4. The method according to claim 1, wherein said poly(farnesene) polymer present in said composition (C) has a number-average molecular weight of greater than or equal to 20,000 g/mol and less than or equal to 90,000 g/mol, and in particular a number-average molecular weight of greater than or equal to 40,000 g/mol and less than or equal to 80,000 g/mol.

5. The method according to claim 4, wherein said poly(farnesene) polymer present in said composition (C) is totally hydrogenated, functionalized with hydroxyl radicals and has a number-average molecular weight equal to 71,000 g/mol.

6. The method according to claim 1, wherein said composition (C) comprises, for 100% of the weight thereof,
40% by weight of a totally hydrogenated poly(farnesene) polymer functionalized with hydroxyl groups and having a number-average molecular weight equal to 71,000, and
60% by weight of a mixture (M) of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms.

7. The method according to claim 1, wherein said composition (C) is implemented in a cosmetic formulation (F) comprising from 0.2% to 40% by weight thereof for 100% of the weight thereof.

8. The method according to claim 7, wherein said formulation (F) is an emulsion (E) of an aqueous phase (A) and a fatty phase (G) comprising, for 100% of the weight thereof:
from 50% to 98% by weight of said aqueous phase (A), and
from 2% to 50% by weight of said fatty phase (G),
said fatty phase (G) comprising, for 100% of the weight thereof:
from 10% to 80% by weight of said fatty composition (C),
from 0.5% to 20% by weight of an emulsifying surfactant (S) or a mixture of emulsifying surfactants (Ms) of the oil-in-water or water-in-oil type, and
from 0% to 89.5% by weight of an oil or a plurality of oils and/or a wax, said wax being different from said composition (C).

9. The method according to claim 7, wherein said composition (C) is implemented in a cosmetic formulation (F) comprising from 1% to 10% by weight thereof and more particularly from 2% to 5% by weight, for 100% of the weight thereof.

10. A method for drying the hair, wherein it comprises the following consecutive steps:
a step a) of applying to the hair a cosmetic formulation (F) comprising, for 100% of the weight thereof, from 0.2% to 40% by weight of a composition (C), said composition (C) comprising, for 100% of the weight thereof,
from 10% by weight to 90% by weight of at least one poly(farnesene) polymer having a number-average molecular weight of greater than or equal to 10,000 g/mol and less than or equal to 120,000 g/mol, said poly(farnesene) polymer being partially or totally hydrogenated and/or functionalized with hydroxyl radicals, and
from 10% by weight to 90% by weight of a mixture (M) of hydrocarbons in which at least 94% by weight of said hydrocarbons comprise from fifteen to nineteen carbon atoms,
optionally a step b) during which, at the end of step a), the hair is left to rest at room temperature for a duration of 5 to 60 minutes, then
a step c) of drying the hair at the end of step a) or optionally at the end of step b), by means of a heating appliance such as a hair dryer or heated hood.

11. The method according to claim 10, wherein said mixture (M) of hydrocarbons present in the composition (C) contained in the formulation (F) implemented in step a) is a mixture of saturated hydrocarbons comprising, for 100% of the weight thereof:
from 15% to 20% by weight of alkanes (linear, isoalkanes and cycloalkanes) comprising 17 carbon atoms,
from 70% to 75% by weight of alkanes (linear, isoalkanes and cycloalkanes) comprising 18 carbon atoms, and
from 4% to 6% by weight of alkanes (linear, isoalkanes and cycloalkanes) comprising 19 carbon atoms.

12. The method according to claim 11 wherein said mixture (M) of hydrocarbons present in the composition (C) contained in the formulation (F) implemented in step a) is a mixture of saturated hydrocarbons comprising, for 100% of the weight, thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms.

13. The method according to claim 10, wherein said poly(farnesene) polymer present in the composition (C) contained in the formulation (F) implemented in step a) has a number-average molecular weight of greater than or equal to 20,000 g/mol and less than or equal to 90,000 g/mol, and in particular a number-average molecular weight of greater than or equal to 40,000 g/mol and less than or equal to 80,000 g/mol.

14. The method according to claim 13, wherein said poly(farnesene) polymer present in said composition (C) contained in the formulation (F) implemented in step a) is totally hydrogenated, functionalized with hydroxyl radicals and has a number-average molecular weight equal to 71,000 g/mol.

15. The method according to claim 10, wherein said composition (C) contained in the formulation (F) used in step a) comprises, for 100% of the weight thereof,
40% by weight of a totally hydrogenated poly(farnesene) polymer functionalized with hydroxyl groups and having a number-average molecular weight equal to 71,000, and
60% by weight of a mixture (M) of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms.

16. The method according to claim 10, wherein said composition (C) is implemented in an emulsion (E) of an aqueous phase (A) and a fatty phase (G) comprising, for 100% of the weight thereof:
from 50% to 98% by weight of said aqueous phase (A), and
from 2% to 50% by weight of said fatty phase (G),
said fatty phase (G) comprising, for 100% of the weight thereof:
from 10% to 80% by weight of said fatty composition (C),
from 0.5% to 20% by weight of an emulsifying surfactant (S) or a mixture of emulsifying surfactants (MS) of the oil-in-water or water-in-oil type, and
from 0% to 89.5% by weight of an oil or a plurality of oils and/or a wax, said wax being different from said composition (C).

17. The method according to claim 10, wherein said composition (C) is implemented in a cosmetic formulation (F) comprising, for 100% of the weight thereof, from 1% to 10% thereof, and more particularly from 2% to 5% by weight thereof.

18. The method for shaping the hair, wherein it comprises the following consecutive steps:
a step d) of applying, to dry or wet hair, a cosmetic formulation (F) comprising, for 100% of the weight thereof, 0.2% to 40% by weight of a composition (C) comprising, for 100% of the weight thereof,
from 10% by weight to 90% by weight of at least one poly(farnesene) polymer having a number-average molecular weight of greater than or equal to 10,000 g/mol and less than or equal to 120,000 g/mol, said poly(farnesene) polymer being partially or totally hydrogenated and/or functionalized with hydroxyl radicals, and
from 10% by weight to 90% by weight of a mixture (M) of hydrocarbons in which at least 94% by weight of said hydrocarbons comprise from fifteen to nineteen carbon atoms,
optionally a step e) during which, at the end of step d), the hair is left to rest at room temperature for a duration of 5 to 60 minutes, then,
a step f) of shaping the hair at the end of step d or optionally at the end of step e), by means of a heating appliance such as a straightening iron or curling tongs.

19. The method according to claim 18 wherein said mixture (M) of hydrocarbons present in the composition (C) contained in the formulation (F) implemented in step d) is a mixture of saturated hydrocarbons comprising, for 100% of the weight thereof:
from 15% to 20% by weight of alkanes (linear, isoalkanes and cycloalkanes) comprising 17 carbon atoms,
from 70% to 75% by weight of alkanes (linear, isoalkanes and cycloalkanes) comprising 18 carbon atoms, and
from 4% to 6% by weight of alkanes (linear, isoalkanes and cycloalkanes) comprising 19 carbon atoms.

20. The method according to claim 19, wherein said mixture (M) of hydrocarbons present in the composition (C) contained in the formulation (F) implemented in step d) is a mixture of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms.

21. The method according to claim 18, wherein said poly(farnesene) polymer present in the composition (C) contained in the formulation (F) implemented in step d) has a number-average molecular weight of greater than or equal to 20,000 g/mol and less than or equal to 90,000 g/mol, and in particular a number-average molecular weight of greater than or equal to 40,000 g/mol and less than or equal to 80,000 g/mol.

22. The method according to claim 21, wherein said poly(farnesene) polymer present in said composition (C) contained in the formulation (F) implemented in step d) is totally hydrogenated, functionalized with hydroxyl radicals and has a number-average molecular weight equal to 71,000 g/mol.

23. The method according to claim 18, wherein said composition (C) contained in the formulation (F) implemented in step a) comprises, for 100% of the weight thereof,
40% by weight of a totally hydrogenated poly(farnesene) polymer functionalized with hydroxyl groups and having a number-average molecular weight equal to 71,000, and
60% by weight of a mixture (M) of saturated hydrocarbons comprising, for 100% of the weight thereof:
3.8% linear alkanes comprising from fifteen to nineteen carbon atoms,
96.2% isoalkanes comprising from fifteen to nineteen carbon atoms, and
less than 0.1% cycloalkanes comprising from fifteen to nineteen carbon atoms, and in which:
17.1% by weight of alkanes (linear, isoalkanes and cycloalkanes) comprise 17 carbon atoms,
72.5% by weight of alkanes (linear, isoalkanes and cycloalkanes) comprise 18 carbon atoms, and
4.7% by weight of alkanes (linear, isoalkanes and cycloalkanes) comprise 19 carbon atoms.

24. The method according to claim 18, wherein said composition (C) is implemented in an emulsion (E) of an aqueous phase (A) and a fatty phase (G) comprising, for 100% of the weight thereof:
from 50% to 98% by weight of said aqueous phase (A), and
from 2% to 50% by weight of said fatty phase (G),
said fatty phase (G) comprising, for 100% of the weight thereof:
from 10% to 80% by weight of said fatty composition (C),
from 0.5% to 20% by weight of an emulsifying surfactant (S) or a mixture of emulsifying surfactants (MS) of the oil-in-water or water-in-oil type, and
from 0% to 89.5% by weight of an oil or a plurality of oils and/or a wax, said wax being different from said composition (C).

25. The method according to claim 18, wherein said composition (C) is implemented in a cosmetic formulation (F) comprising, for 100% of the weight thereof, from 1% to 10% thereof, and more particularly from 2% to 5% by weight thereof.
